(19) United States
(12) Patent Application Publication
(10) Pub. No.: US 2025/0057648 A1
(43) Pub. Date: Feb. 20, 2025

(54) Heart Valve

(71) Applicant: TAVR AI Pty Ltd., Norman Park (AU)

(72) Inventor: Ryan Markham, Queensland (AU)

(21) Appl. No.: 18/717,667

(22) PCT Filed: Dec. 8, 2022
(86) PCT No.: PCT/AU2022/051473

§ 371 Date: Jun. 7, 2024

(30) Foreign Application Priority Data
Dec. 9, 2021 (AU) 2021903987

(51) Int. Cl. A61F 2/24; A61F 2/88

(52) U.S. Cl. CPC A61F 2/2418; A61F 2/2427; A61F 2/88; A61F 2210/0014; A61F 2230/0091

(57) ABSTRACT

A valve for repair of a regurgitant orifice between an atrium and a ventricle in a heart is disclosed. The valve includes a frame comprising a helical coil having at least two turns and defining a central opening. One or more leaflets are attached to the helical coil and extend into the central opening. When the valve is implanted, the valve is configured to clamp native leaflet tissue of the heart between adjacent turns of the helical coil. Also disclosed is a surgical kit including the valve and a method of delivering the valve to a heart.

### CROSS-REFERENCE TO RELATED APPLICATIONS

This application is the United States national phase of International Patent Application No. PCT/AU2022/051473 filed Dec. 8, 2022, and claims priority to Australian Patent Application No. 2021903987 filed Dec. 9, 2021, the disclosures of which are hereby incorporated by reference in their entireties.

### BACKGROUND OF THE INVENTION

### Field of the Invention

The present disclosure is related to the field of heart valves and more particularly concerns valves for repair and/or replacement of a regurgitant orifice between an atrium and a ventricle.

### Description of Related Art

The heart includes four valves; the aortic, mitral, tricuspid and pulmonary valves, which regulate the direction of blood flow through the chambers of the heart. The mitral valve regulates blood flow from the left atrium to the left ventricle of the heart. It has a saddle-shaped annulus and two valve leaflets. The tricuspid valve allows blood to flow from the right atrium to the right ventricle. It also has a saddle-shaped annulus and three valve leaflets. The leaflets open to allow blood to flow through the valve from the atrium to the ventricle during diastole and close to prevent backflow of blood from the ventricle to the atrium during systole.

Sometimes, a heart valve may not function correctly. For example, the valve may be defective or may have become damaged due age, disease or other degeneration. Regurgitation (also known as leaking) is a condition in which the valve leaflets do not close appropriately during systole, allowing blood to flow backward into the atrium. Based on the severity, surgical intervention may be required to repair or replace the valve. Mitral valve regurgitation is the most common valvular heart disease, impacting 3.5% of the population, while tricuspid valve regurgitation has a prevalence of 2.5%. Left untreated, severe mitral and tricuspid regurgitation can be fatal with a mortality rate exceeding 50% within 12 months.

Many patients with mitral or tricuspid valve regurgitation do not tolerate open heart surgery. Instead, transcatheter heart therapies which involve minimally invasive procedures via venous access may be performed to repair or treat diseased heart valves, avoiding the need for open heart surgery which carries higher risk involving sternotomy and cardiopulmonary bypass. However, there are currently limited effective transcatheter mitral and tricuspid therapies (TMTT), with current generation devices unable to be used in a significant proportion of patients due to prohibitive anatomy. Furthermore to date there has not been demonstrated superiority of outcome for TMTT as compared to open heart surgery. As such, there is a need for a way to perform lower risk mitral or tricuspid valve repair in patients with mitral or tricuspid regurgitation that can be used in a wide variety of patient anatomy and with superiority of outcome to conventional open heart surgery.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY OF THE INVENTION

According to one aspect of the present disclosure, there is provided a valve for repair of a regurgitant orifice between an atrium and a ventricle in a heart, the valve comprising:
a frame comprising a helical coil having at least two turns and defining a central opening; and
one or more leaflets attached to the helical coil and extending into the central opening,
wherein, when the valve is implanted, the valve is configured to clamp native leaflet tissue of the heart between adjacent turns of the helical coil.

In some embodiments, the helical coil may be formed from a shape memory material. For example, the shape memory material may be Nitinol. The shape memory material may be configured to have a transformation temperature of between about −20° C. and about 30° C. For example, the transformation temperature may be about −20° C., about −15° C., about −10° C., about −5° C., about 0° C., about 5° C., about 10° C., about 15° C., about 20° C., about 25° C., about 30° C., about 35° C., about 40° C. or more. Typically, the transformation temperature is between 20° C. and 30° C.

The helical coil may be formed from a wire. The wire may have a substantially circular cross-section. A diameter of the wire may be between about 0.3 mm and about 1.5 mm, for example, about 0.5 mm or about 1.0 mm. In some embodiments, the diameter of the wire may be about 0.3 mm, about 0.5 mm, about 0.7 mm, about 0.9 mm, about 1.1 mm, about 1.3 mm, about 1.5 mm or more.

In some embodiments, the helical coil may have more than two turns, For example, the helical coil may have three or more turns. In some embodiments, the helical coil may have an integer or a non-integer number of turns. For example, the body may comprise about 2.5, about 3, about 3.25, about 3.5, about 3.75 about 4, about 4.5, about 5 or more turns of the helical coil.

The frame may be configurable between an axially elongated configuration and a collapsed configuration.

In the collapsed configuration, the helical coil typically defines an annular body defining the central opening. In the collapsed configuration the annular body has a collapsed configuration diameter. In the elongated configuration, the diameter of the annular body may be less than the collapsed configuration diameter. For example, upon elongation of the helical coil, the turns of the helical coil may be stretched open in the axial direction, in the manner of stretching a spring. As the helical coil is stretched axially, a diameter of the helical coil (and thus a diameter of the central opening) may be reduced. For example, the diameter of the body in the axially elongated configuration may be reduced by about 20%, about 30%, about 40%, about 50%, about 60%, about 70% or more from collapsed configuration diameter. As will be appreciated, the extent of diameter reduction in the axially elongated configuration may depend on the extent to which the helical coil is axially stretched. The degree of diameter reduction may therefore be adjustable by a user.

In some embodiments, the collapsed configuration diameter of the helical coil may be selected based on a size of the native valve. Additionally or alternatively, the collapsed configuration diameter may be selected based on a size of the regurgitant orifice. Repair of the regurgitant orifice may comprise covering the regurgitant orifice with the valve. In some embodiments, where the regurgitant orifice is relatively large, for example, repair of the regurgitant orifice may comprise replacement of substantially the entire native valve.

In some embodiments, a maximum value of the collapsed configuration diameter may be between about 10 mm and about 100 mm, for example, about 16 mm, or about 25 mm. In other embodiments, a maximum value of the collapsed configuration diameter may be about 10 mm, about 12 mm, about 14 mm, about 16 mm, about 18 mm, about 20 mm, about 22 mm, about 24 mm, about 26 mm, about 28 mm, about 30 mm, about 35 mm, about 40 mm, about 50 mm, about 60 mm, about 70 mm, about 80 mm, about 90 mm, about 100 mm or more. In some embodiments, the repair may be effected by the valve clamping to native leaflet tissue such that the valve covers the regurgitant orifice. In such embodiments, a maximum value of the collapsed configuration diameter of the helical coil may be selected to be smaller than a diameter of the native heart valve, but larger than a diameter of the regurgitant orifice. In some embodiments, the valve may be considered to provide a replacement of the entire native valve. This may be the case where the regurgitant orifice is relatively large, involving all of the native valve leaflets for example. In such embodiments, the valve may correspondingly have a relatively large diameter, for example 60 mm or more, which may replace the entire native mitral or tricuspid valve.

In some embodiments, the helical coil is resiliently biased into the collapsed configuration. For example, in embodiments where the coil is formed from a shape memory material, the shape memory material may be configured to return to the collapsed configuration at or above a predetermined transformation temperature.

The helical coil may be configured to have a tight pitch when in the collapsed configuration. For example, in the collapsed configuration, adjacent turns of the helical coil may abut one another. The helical coil may be configured such that, in the collapsed configuration, adjacent turns of the coil clamp against one another with a clamping force. The clamping force may be configured or predetermined to provide secure fixation of the valve to native leaflets of the heart, when implanted. The valve may be configured to be delivered to a native valve of the heart in the elongated configuration and configured to clamp native leaflet tissue of the heart when released to the collapsed configuration.

In some embodiments, the valve may comprise a single leaflet. The leaflet may be configured to abut against one or more reinforcing members or reinforcing elements to close the valve.

In other embodiments, the valve may comprise two leaflets. In other embodiments, the valve may comprise three leaflets. In other embodiments, the valve may have comprise or more leaflets.

The leaflets may be formed from a material selected from a group consisting of animal leaflet tissue material (for example, bovine or porcine leaflet tissue), pericardial tissue or polymeric material. One or more of the leaflets may be formed from a polymeric material (such as PTFE, for example) in combination with a reinforcing material. The reinforcing material may be a mesh structure, for example. The reinforcing material may be attached to and/or embedded in the polymeric material. The reinforcing material may be a nitinol mesh, for example. The leaflets may be formed from the same material as each other. Alternatively, the leaflets may be formed from different material to each other. In some examples, an atrial side leaflet may be formed from a polymeric material, while the other leaflet (or leaflets) may be formed from tissue.

The leaflets may be attached to the helical coil around a perimeter of the central opening. The leaflets may be directly attached to the helical coil, using sutures for example. In other embodiments, alternative fixation methods may be used. In some embodiments, the leaflets are attached to an inner surface of the annular body defined by the helical coil.

The leaflets may be attached to the helical coil (directly or indirectly) such that the leaflets are positioned sequentially along the helical coil. In some embodiments, adjacent leaflets may partially overlap one another at the point of attachment to the wire of the helical coil. In other embodiments, adjacent leaflets may abut one another at the point of attachment to the wire of the helical coil without overlapping at the point of attachment to the wire of the helical coil.

The leaflets may be attached along the wire of the helical coil such that, when the frame is in the axially elongated configuration, the leaflets are also configured in an axially extended configuration. In the axially extended configuration, the leaflets may be substantially separated from one another in an axial direction. For example, in the axially elongated configuration, a leaflet may contact an adjacent leaflet at peripheral region and/or edge (such as at a peripheral point of the leaflet attachment to the wire of the helical coil and/or at a coaptation line) while other regions of the leaflet may be axially separated from the adjacent leaflet.

In some embodiments, the leaflets may be arranged along the wire of the helical coil such that, in the collapsed configuration, the leaflets form an overlapping sequence. That is, the leaflets may be configured to overlap within the central opening. For example, the first leaflet may overlap the second leaflet by an area of about 1%, about 2%, about 3%, about 4%, 5%, about 10%, about 15% or more of a total area of the first leaflet. The leaflets may be configured to overlap to close the central opening when at rest. In some cases, adjacent leaflets may overlap by an area of around 1% of one leaflet and may additionally be sutured together along a coaptation line. The coaptation line may extend a distance of between about 0.5 mm and about 4 mm, about 1 mm and about 3 mm, or about 2 mm, for example.

The leaflets may be configured (for example by overlapping and/or a coaptation line) to inhibit prolapse of the leaflets beyond the annular body in an atrial direction. For example, the leaflets may be arranged such that, when the helical coil is in the collapsed configuration, the natural deflection of the leaflets is towards the ventricular side of the valve.

In some embodiments, the leaflets may be attached to a support. The support may be attached to the helical coil such that the leaflets are positioned around a perimeter of the central opening. In some embodiments, the support may comprise a base. The leaflets may be attached to the base. The base may be attached to the helical coil. The base may be elongate and may extend along a length of the helical coil. The support may additionally and/or alternatively comprise one or more struts. The one or more struts may extend substantially perpendicular to a local portion of the base. One or more of the leaflets may be attached one or more of the struts. For example, the one or more struts may be attached to one or more respective peripheral edges of the leaflets. Adjacent pairs of the leaflets may be attached to a common strut. The support may be formed from a polymeric material, such as PTFE for example. The support may additionally and/or alternatively comprise metal wire.

The leaflets and the struts may move between a closed position and an open position. In the open position, the leaflets and the struts may extend in a ventricular direction to allow fluid to flow through the central opening. In the closed position, the leaflets may substantially cover the central opening. When implanted in a heart, the leaflets may move in a ventricular direction during diastole, that is, into the open position, to allow blood to flow from the atrium into the ventricle. The leaflets and struts may be configured such that the leaflets are inhibited from prolapsing in an atrial direction during systole, such that the valve inhibits regurgitant blood flow from the ventricle to the atrium during systole.

In some embodiments, the valve may comprise a first leaflet and a second leaflet. The first leaflet and the second leaflet may be unequal in size and/or unequal in shape. In some embodiments, the first leaflet may have a crescent-shaped portion extending into the central opening. In some embodiments, the second leaflet may include a gibbous-shaped portion extending into the central opening. The second leaflet may extend across a greater area of the central opening than the first leaflet. The attachment of the first leaflet to the helical coil may extend along a greater perimeter length of the central opening than the second leaflet. As such, a ratio of the leaflet area to attachment perimeter length may be greater for the second leaflet than for the first leaflet. In such embodiments, the first leaflet may have increased stiffness, or may be more resistant to movement, than the second leaflet. As an example, in some embodiments, a crescent-shaped portion of the first leaflet may extend across about one-third of an area of the central opening, while a gibbous-shaped portion of the second leaflet may extend across about two-thirds of said area. The attachment of the first leaflet may extend along about two-thirds of a perimeter of the central opening, while the attachment of the second leaflet extends along about one-third of said perimeter. In such embodiments, the crescent-shaped leaflet may have increased stiffness compared to the gibbous shaped leaflet, due to the reduced ratio of the leaflet area to the perimeter attachment length.

The first leaflet may overlap the second leaflet on an atrial side of the valve. The first leaflet may inhibit movement of the second leaflet beyond the annular body in the atrial direction. For example, in embodiments employing a crescent-shaped and a gibbous-shaped leaflet, such as described above, the stiffer, crescent-shaped leaflet may be provided on the atrial side of the valve, inhibiting prolapse of the gibbous-shaped leaflet in an atrial direction during systole.

In some embodiments, the first leaflet and the second leaflet may both move in a ventricular direction during diastole. The second leaflet may have a greater range of movement than the first leaflet (when the first leaflet is a stiffer leaflet, for example). In some embodiments, the first leaflet may remain substantially stationary during systole and diastole. In such embodiments, the first leaflet may be considered to function as a reinforcing element for the second leaflet.

In some embodiments, the frame further comprises a reinforcing member. The reinforcing member may extend into the central opening from a perimeter of the annular body. The reinforcing member may be positioned on an atrial side of the leaflets. The reinforcing member may inhibit prolapse of the leaflets beyond the body in an atrial direction.

The reinforcing member may extend substantially within a plane defined by a superior turn (for example, the last turn on the atrial side of the valve) of the helical coil. For example, in some embodiments, the reinforcing member may be a continuation of the material of the annular body. The reinforcing member may be positioned such that it abuts an atrial side of one or more of the leaflets. In one embodiment, the reinforcing member is positioned in proximity of the leaflet closest to the atrial side of the valve. The reinforcing member may be a continuation of the wire of the helical coil, for example, a continuation of the superior turn of the helical coil. The reinforcing member may spiral inwardly from the perimeter of the annular body into the central opening. The spiral of the reinforcement member may comprise two complete turns. In some embodiments, the spiral of the reinforcement member may have a gradually decrementing diameter toward a centre point of the central opening. In some embodiments, the spiral of the reinforcement member may approximate a Fibonacci curve. However, other ratios of decrementing arc diameter may be used. In other embodiments, the reinforcing member could be any number of configurations which extend from the perimeter of the annular body into the central opening such as to prevent prolapse of the leaflets in the atrial direction.

In some embodiments, the frame may include an attachment member for attachment to a delivery device. Attachment of the attachment member to the delivery device may axially lock the attachment member to the delivery device, or inhibit relative axial movement between the attachment member and the delivery device.

The attachment member may be provided at a distal end of the frame. When in situ, the distal end of the frame is located closest to the ventricle. The attachment member may be fixed to the distal end of the frame. For example, the attachment member may be welded or otherwise bonded to the distal end of the frame. In other embodiments a distal end of the frame (e.g. of the helical coil) may be wrapped around a portion of the attachment member to secure the attachment member to the frame. For example, the attachment member may comprise a body portion and a distal end of the helical coil may wrap around the body portion. In some embodiments, the attachment member may comprise a case for receiving, retaining, and/or substantially covering the body portion and the distal end of the frame.

The distal end of the frame may include an arm extending inwardly from a perimeter of the annular body. The arm may be configured as a spiral having a gradually decreasing diameter. For example, the arm may be a continuation of a distal turn of the helical coil. The arm may extend distally from the annular body such that the arm allows for movement of the leaflets in the ventricular direction.

The attachment member may include a threaded portion configured to engage a correspondingly threaded portion of a delivery device. The threaded portion may comprise an internal thread and/or an external thread. The threaded portion may be provided on an outer surface of the attachment member. The threaded portion may be provided on a case of the attachment member, for example. In other embodiments, the attachment member may engage the delivery device by other means.

In some embodiments, the frame may further include an engagement portion at a proximal end of the helical coil. The engagement portion may include an aperture configured to be engaged by one or more sutures or wires. For example, the engagement portion may include a loop, a hook or a ring. The engagement portion may be configured to be engaged by the one or more sutures or wires by looping the one or more sutures or wires through the loop, hook or ring. When the delivery device is attached to the attachment member, tensile force may be applied to the engagement portion to retract the proximal end of the coil, thereby to configure the valve in the axially elongated configuration for delivery to the heart. In some embodiments, the engagement member is attached to the reinforcing member. The attachment member may be positioned at a location on the reinforcing member which is centrally located in the central opening when the helical coil in its collapsed configuration.

According to another aspect of the present disclosure, there is provided a kit, comprising:
a valve for repair of a regurgitant orifice between an atrium and a ventricle in a heart, the valve comprising:
   a frame comprising a helical coil having at least two turns and defining a central opening; and
   one or more leaflets attached to the helical coil and extending into the central opening,
wherein, when the valve is implanted, the valve is configured to clamp native leaflet tissue of the heart between adjacent turns of the helical coil; and
a delivery device.

In some embodiments, the kit may further comprise a catheter. The kit may further comprise surgical tools and/or equipment for performing the procedure. The kit may further comprise instructions for preparing and/or implanting the valve In some embodiments, the valve may be provided in a container. The container may further contain a preserving material. For example, the valve may be provided in a container of formaldehyde prior to implantation.

According to another aspect of the present disclosure, there is provided a method of delivering a valve according to the present disclosure, for repair of a regurgitant orifice between an atrium and a ventricle in a patient's heart, the method comprising:
attaching the valve to a delivery device;
configuring the valve body in an axially elongated configuration;
delivering the valve to the heart through vasculature of the patient;
positioning the valve across the regurgitant orifice; and
configuring the valve in a collapsed configuration such that native leaflet tissue is clamped between adjacent turns of the helical coil to secure the valve across the regurgitant orifice.

The valve may be positioned across the regurgitant orifice with the valve leaflets on the ventricular and/or the atrial side of the regurgitant orifice.

The delivery device and the valve may be configured for delivery to the patient's heart via a catheter. The catheter may be steerable. The catheter may be configured to substantially contain the valve in its axially elongated configuration, along the delivery rod.

The method may further comprise steps of assembling the delivery device, one or more retraction threads or wires and delivery catheter. For example, a delivery device, Nitinol retraction wire/thread and steerable delivery catheter may be provided in respective, separate packets, and may be assembled prior to surgery. The valve may then be attached to the delivery device, elongated axially by retracting the thread and sheathed in the catheter prior to delivery to the heart.

In some embodiments, the delivery device may comprise an semi-flexible rod having an attachment member at a distal end. The attachment member may be configured to attach to a distal end of the helical coil. For example, a distal end of the delivery device may have a threaded portion. The threaded portion may have a thread corresponding to a threaded portion of the attachment member of the valve. In other embodiments, however, the attachment member of the delivery device and the attachment member of the valve may not be threaded, but may be releasably engageable by other suitable attachment means.

In some embodiments, configuring the valve in an axially elongated configuration may comprise retracting a proximal end of the helical coil relative to the distal end, thereby to elongate the coil. For example, in some embodiments, the method includes attaching one or more sutures or wires to the engagement portion of the valve (for example, by looping a thread or thin Nitinol wire around the proximal enforcement member) and applying force to retract the proximal end of the helical coil. In some embodiments, the valve may be configured in the axially elongated position below a transformation temperature of the shape memory material, such that the valve remains in the axially elongated configuration without continuous application of retracting force. For example, the valve may be subjected to decreased temperatures (such as in an ice water bath or other relatively medium below the transformation temperature of the shape memory material) prior to delivery to the patient.

Configuring the valve in a collapsed configuration may comprise releasing the retracted proximal end of the helical coil, by releasing the retractile force on the proximal end of the coil. Additionally or alternatively, configuring the valve in a collapsed configuration may comprise subjecting the valve to temperatures above a transformation temperature of the shape memory material, such that the shape memory properties of the shape memory material return the valve to the collapsed configuration. As the valve returns to the collapsed configuration, native leaflet tissue may be clamped between turns of the coil, securing the valve across the regurgitant orifice.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE DRAWINGS

The terms Fig., Figs., Figure, and Figures are used interchangeably in the specification to refer to the corresponding figures in the drawings.

By way of example only, embodiments are now described with reference to the accompanying drawings, in which:

FIG. 1 is a perspective view of a valve according to one embodiment of the present disclosure;

FIG. 2 is a perspective view of a frame of a valve according to another embodiment of the present disclosure;

FIG. 3 is a top (atrial) view of the frame of the valve of FIG. 2;

FIG. 4A is a top (atrial) view of a frame of the valve of FIG. 1;

FIG. 4B is a front view of the frame of the valve of FIG. 4A;

FIG. 5 is a perspective view of leaflets of the valve of FIG. 1;

FIG. 6 is a bottom (ventricular) view of the valve of FIG. 1;

FIG. 7 is a top (atrial) view of the valve of FIG. 1;

FIG. 8 is a bottom (ventricular) perspective view of a valve according to another embodiment of the present disclosure;

FIG. 9 is a top (atrial) perspective view of the valve of FIG. 8;

FIG. 10A is a diagram of a first, crescent-shaped leaflet of the valve of FIG. 8;

FIG. 10B is a diagram of a second, gibbous-shaped leaflet of the valve of FIG. 8;

FIG. 10C is a diagram of first and second leaflets of the valve of FIG. 8;

FIG. 11A is a top (atrial) view of the valve of FIG. 8, showing the leaflets in a closed position;

FIG. 11B is a bottom (ventricular) view of the valve of FIG. 8, showing the leaflets in the closed position;

FIG. 12A is a top (atrial) view of the valve of FIG. 8, showing the leaflets in an open position;

FIG. 12B is a bottom (ventricular) view of the valve of FIG. 8, showing the leaflets in the open position;

FIG. 13 is a perspective view of the valve of FIG. 8 attached to a delivery device;

FIG. 14 is a front view of the valve of FIG. 8 attached to the delivery device of FIG. 13;

FIG. 15A is a front view of the valve of FIG. 8 attached to the delivery device of FIG. 13 and in an axially elongated configuration;

FIG. 15B is a front view of the valve of FIG. 1 attached to the delivery device of FIG. 13 and in an axially elongated configuration;

FIG. 16A and 16B are a front view and top view, respectively, of a valve according to another embodiment of the present disclosure showing a coaptation line;

FIG. 17A is a partial schematic illustration of a heart valve according to another embodiment of the present disclosure, showing leaflets and a support attached to a helical coil in an axially elongated configuration.

FIG. 17B is a perspective view showing the leaflets and support of FIG. 17B in a collapsed configuration with the leaflets in an open position;

FIG. 17C is a top view showing the leaflets and support of FIG. 17B in a collapsed configuration with the leaflets in a closed position;

FIG. 18A is a partial perspective view showing a distal region of a delivery member and a heart valve according to an embodiment of the present disclosure;

FIG. 18B is a perspective view showing a body of the delivery member of FIG. 18A;

FIG. 18C is a partial cross-section front view showing a distal region of the delivery member of FIG. 18A;

FIG. 18D is a front view showing the delivery member of FIG. 18A and a frame of the heart valve of FIG. 18A with a thread engaging the engagement portion; and

FIG. 19 is a flow diagram illustrating steps in a method of implanting a heart valve according to the present disclosure.

### DESCRIPTION OF THE INVENTION

A valve according to the present disclosure, for repair of a regurgitant orifice between an atrium and a ventricle in a heart, is shown in the drawings as 100. The valve 100 comprises a frame 200 and leaflets 300. The frame 200 includes a helical coil 220 defining a central opening 230. The leaflets 300 are attached to the coil 220 and extend into the central opening 230. When the valve is implanted, the valve 100 is configured to clamp native leaflet tissue of the heart between adjacent turns of the coil 220.

In the illustrated embodiment, the helical coil 220 has about 3.25 turns. In other embodiments, however, the coil 220 may have a greater or lesser number of turns. For example, the helical coil may have about 2, about 2.5, about 2.75, about 3, about 3.25, about 3.5, about 3.75, about 4 or more turns.

The helical coil 220 is depicted as comprising an annular body 210 which has a diameter (D) based on a size of the regurgitant orifice and location of adjacent native leaflet tissue to be clamped. The diameter D may be selected to be larger than a maximum width of the regurgitant orifice. For example, for a regurgitant orifice of about 10 mm, a diameter of the annular body 210 may be about 25 mm. The valve 100 may be configured such that, when clamped to the native leaflet tissue, the valve 100 covers the regurgitant orifice. The valve 100 may augment the native leaflet tissue and/or function in combination with the native leaflet tissue to inhibit or reduce regurgitant blood flow from the ventricle to the atrium during systole. As the valve 100 is configured to clamp onto the native leaflet tissue, the valve 100 may maintain an existing diameter of the native valve.

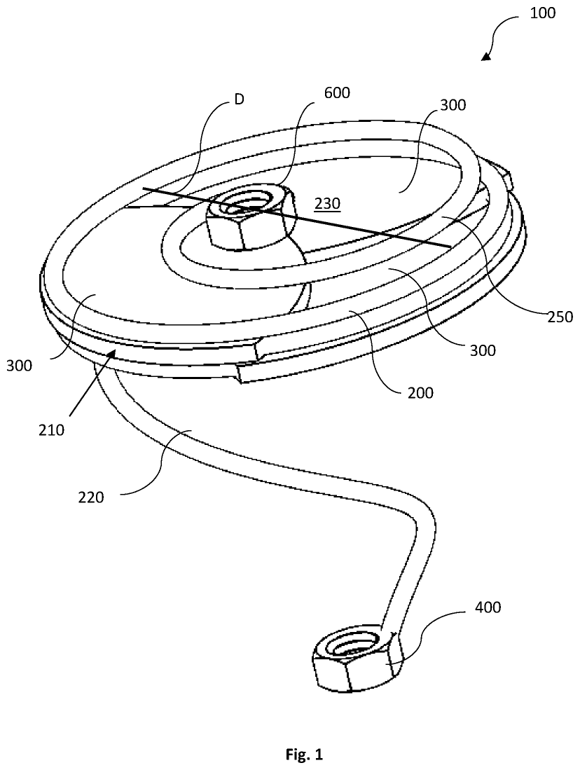

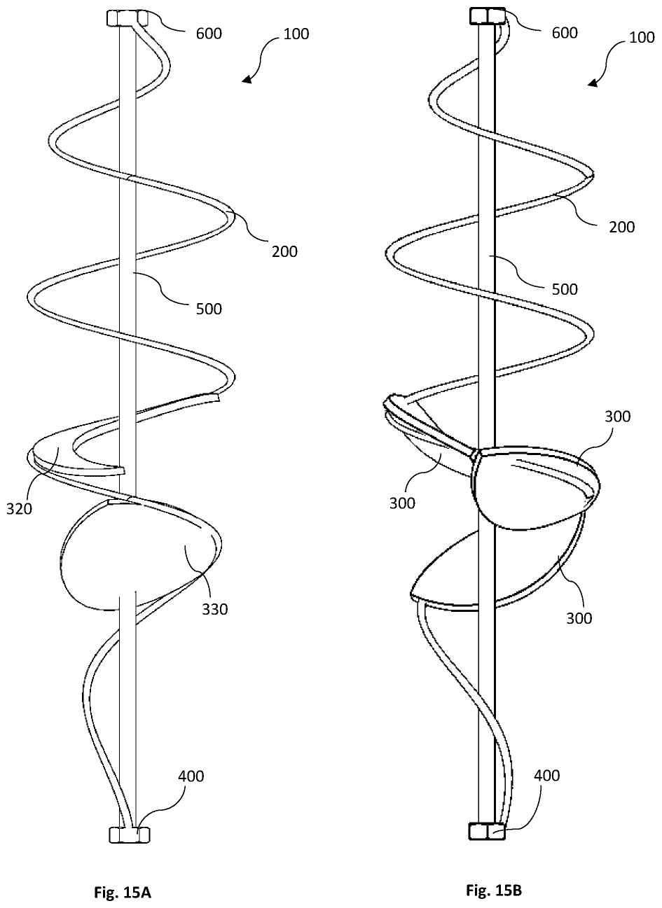

The annular body 210 is configurable between a collapsed configuration, as shown in at least FIG. 1, and an axially elongated configuration, as shown in at least FIGS. 15A and 15B. In the axially elongated configuration, a diameter of the annular body 210 is reduced relative to the diameter in the collapsed configuration. In the illustrated embodiments, the diameter of the annular body 210 is reduced by about 50% in the axially elongated configuration. For example, the diameter may be reduced to about 12.5 mm in the axially elongated configuration. Reduction of the diameter of the annular body 210 allows for transcatheter insertion of the valve 100 through peripheral vasculature of the patient.

The helical coil 220 is resiliently biased into the collapsed configuration. Where the helical coil 220 is formed from a shape memory material, such as Nitinol the helical coil takes on the annular collapsed configuration below a predetermined transformation temperature. In some embodiments, the shape memory properties of the material of the coil 220 may be used to configure the coil in the collapsed and/or axially elongated configurations. For example, the coil 220 may be subjected to a temperature below its transformation temperature such that it may be retained in the axially elongated configuration without continual retractile force. The coil may be subjected to a temperature above the transformation temperature (for example, body temperature) when it is desirable for the coil to be again biased into the collapsed configuration. Alternatively, in some embodiments, the valve may utilise the superelastic properties of Nitinol to allow the coil to configured between the axially elongated and collapsed configurations.

In the embodiment depicted in FIGS. 1,5-7 and 15B, the valve 100 includes three leaflets 300. Each leaflet includes a respective base region 301 and body portion 310. The leaflets 300 may be attached (e.g. fixed) directly to the helical coil 220 along the base region 301. For example the leaflets may be attached with sutures to the helical coil 220 sequentially around an inner diameter of the perimeter of the central opening 230. Adjacent leaflets 300 abut one another at the point of attachment to the wire of the helical coil 220 without overlapping at the point of attachment to the helical coil. In other embodiments, the leaflets 300 may partially overlap at the point of attachment to the helical coil 220.

The leaflets 300 are configured to move between a closed position and an open position when the frame 200 is in the collapsed configuration. In the closed position, the leaflets 300 extend in substantially parallel planes to the other leaflets 300, such that the body portions 310 of the leaflets 300 form an overlapping sequence. The overlapping of the leaflets 300 substantially covers the central opening 230 to inhibit fluid flow therethrough. In the open position, the body portion 310 of each of the leaflets 300 extends in a ventricular direction to allow fluid to flow between the leaflets 300. When implanted in a heart, the leaflets 300 are configured to move in a ventricular direction during diastole, that is, into the open position, to allow blood to flow from the atrium into the ventricle. The overlapping of the leaflets 300 is configured such that the leaflets 300 are inhibited from prolapsing in an atrial direction during systole. As such, during systole, the leaflets 300 abut one another in the closed position to inhibit regurgitant blood flow from the ventricle to the atrium.

When the frame 200 is in the axially elongated configuration, the body portion 310 of each leaflet may be spaced axially from the body portions 310 of the other leaflets 300, as shown in FIG. 15B, for example. When the frame 200 moves from the axially elongated configuration to the collapsed configuration, the leaflets 300 may be deployed in sequence.

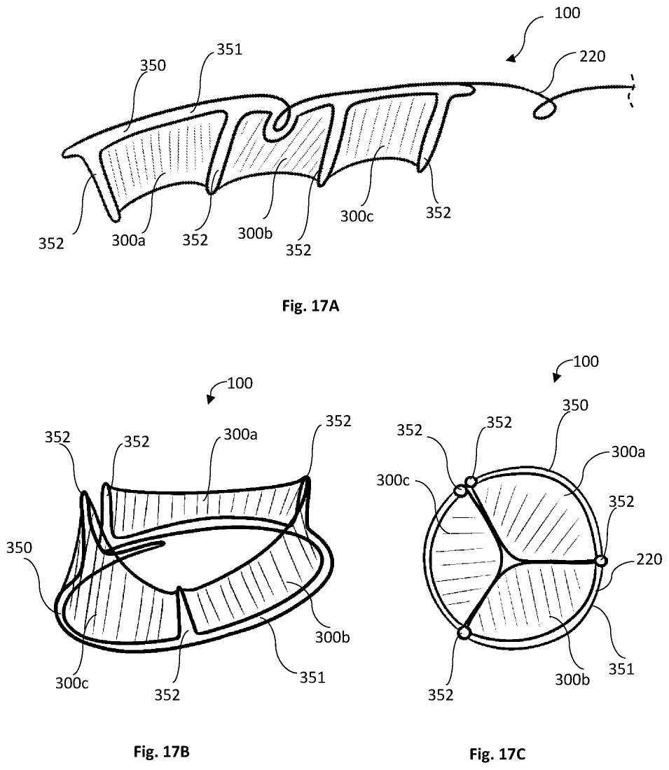

An alternative example of a valve 100 having three leaflets 300a, 300b, 300c is shown in FIGS. 17a-c. In this example, the valve 100 includes a support 350 for attaching the leaflets 300a, 300b, 300c to the helical coil 220. The leaflets 300a, 300b, 300c may be attached to a base 351 of the support 350, which may be attached along a portion of the helical coil 220. The support 350 may position the leaflets 300a, 300b, 300c around an inner diameter of the perimeter of the central opening 230 of the valve 100. FIG. 17a shows the support 350 attached to the helical coil 220, with the helical coil 220 in the axially elongated configuration. The leaflets 300a, 300b, 300c may be positioned adjacent each other along the support 350 (and thus along the helical coil 220).

The support 350 may include one or more struts 352 extending from the base 351. The struts 352 may extend radially inwardly from the base 351 and/or substantially perpendicular to a local portion of the base 351, when the valve 100 is in the closed configuration. The one or more struts 352 may be attached to one or more respective peripheral edges of the leaflets 300a, 300b, 300c. In the example of FIG. 17a, the support 350 includes four struts 352 attached to the peripheral edges of each of the leaflets 300a, 300b, 300c. Adjacent pairs of the leaflets 300a, 300b, 300c may be attached at to a common strut 352, as shown in FIG. 17a. The support 350 may comprise a polymeric material, such as PTFE for example. The support may additionally or alternatively comprise metal wire.

The leaflets 300a, 300b, 300c, along with the struts 352, may move between a closed position and an open position in a similar manner as described above. However, in the example shown in FIG. 17, the leaflets 300a, 300b, 300c may not overlap in the closed position. However, as the edges of leaflets 300a, 300b, 300c are joined by the struts 352, the central opening 230 is substantially covered in the closed position (as shown in FIG. 17c). Thus, in the closed position, the valve 100 inhibits fluid flow through the central opening 230. In the open position, the leaflets 300a, 300b, 300c and the struts 352 extend in a ventricular direction to allow fluid to flow between through the central opening 230. When implanted in a heart, the leaflets 300a, 300b, 300c may move in a ventricular direction during diastole, that is, into the open position, to allow blood to flow from the atrium into the ventricle. The leaflets 300a, 300b, 300c and struts may be configured such that the leaflets 300a, 300b, 300c are inhibited from prolapsing in an atrial direction during systole. As such, during systole, the valve 100 inhibits regurgitant blood flow from the ventricle to the atrium.

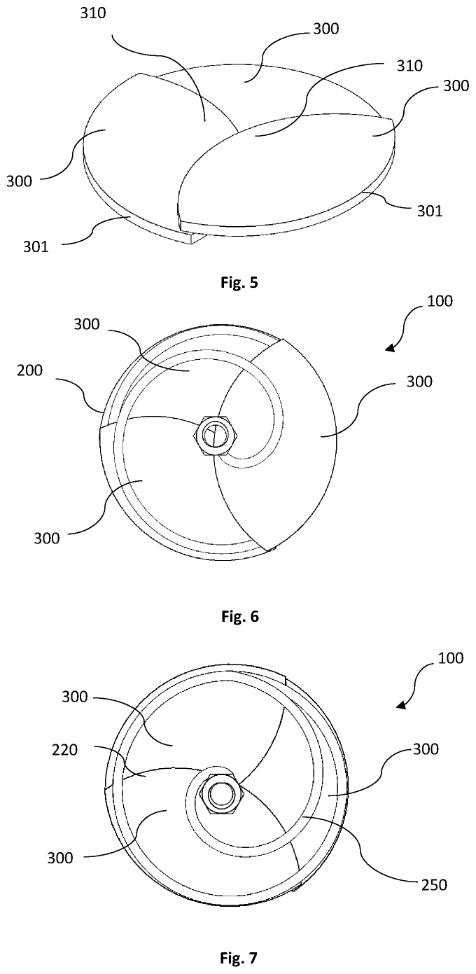

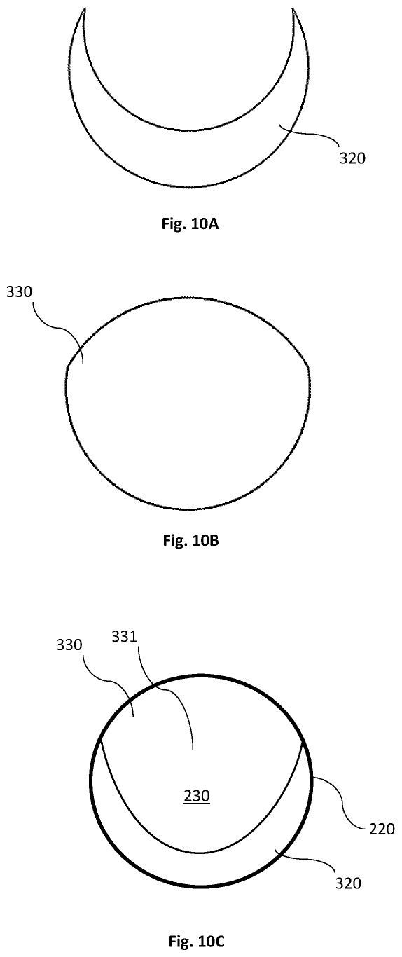

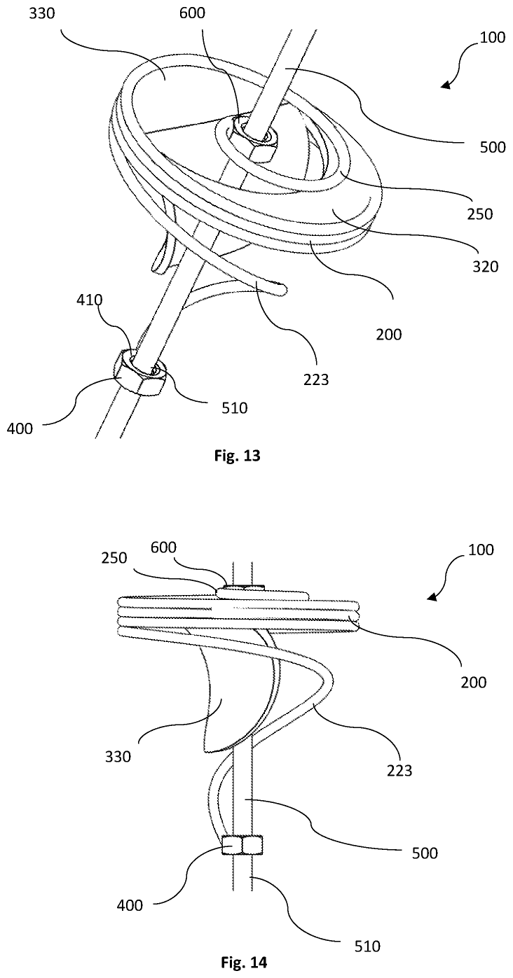

In other, alternative, embodiments, the valve 100 may have two leaflets 300. For example, in the embodiment of FIGS. 8 to 15A, the valve 100 comprises a first leaflet 320 and a second leaflet 330. The first and second leaflets 320, 330 are attached to the coil 220 and share one or more features or functions in common with the leaflets as described above with regards to FIGS. 1 and 5-7. However, in this embodiment, the leaflets 320 330 are unequal in size and shape: as illustrated in FIG. 10, the first leaflet 320 has a crescent shaped portion 321 extending into the central opening, while the second leaflet 330 has a gibbous shaped portion 331 extending into the central opening 230.

The crescent-shaped portion 321 of the first leaflet 320 extends across about one-third of the area of the central opening 230, while the gibbous-shaped portion 331 of the second leaflet 330 extends across about two-thirds of that area. The attachment of the first leaflet 320 extends along about two-thirds of a perimeter of the central opening 230, while the attachment of the second leaflet 330 extends along about one-third of the perimeter. As such, the first, crescent-shaped, leaflet 320 is stiffer and more resistant to movement than the second, gibbous-shaped, leaflet 330. The first leaflet 320 and second leaflet 330 may be formed from the same material (such as animal leaflet tissue material or pericardial tissue, for example). Alternatively, the first leaflet 320 may be formed from a first material and the second leaflet 330 may be formed from a second material, different to the first material. The first material may be less flexible than the second material. In some examples, the first leaflet 320 may be formed from a polymeric material such as PTFE, for example. The first leaflet may include a reinforcing mesh, such as a nitinol mesh for example. The reinforcing mesh may be bonded to and/or embedded in the polymeric material. The use of a polymeric material and a reinforcing material for the first leaflet may provide increased strength and/or stiffness to the first leaflet 320 compared to use of animal or pericardial tissue.

The first and second leaflets 320, 330 overlap within the central opening 230 with the first leaflet 320 positioned on an atrial side of the valve 100. In the illustrated embodiments, the first and second leaflets 320, 330 overlap by about 10%. In other embodiments, the first and second leaflets 320, 330 may overlap to a greater or lesser extent, such as about 1%, about 2%, about 3%, about 4%, about 5%, about 10%, about 15%, about 20%, about 25% or more. The stiffer, crescent-shaped first leaflet 320 may inhibit movement of the second leaflet 330 beyond the body 210 in an atrial direction when in the closed position during systole.

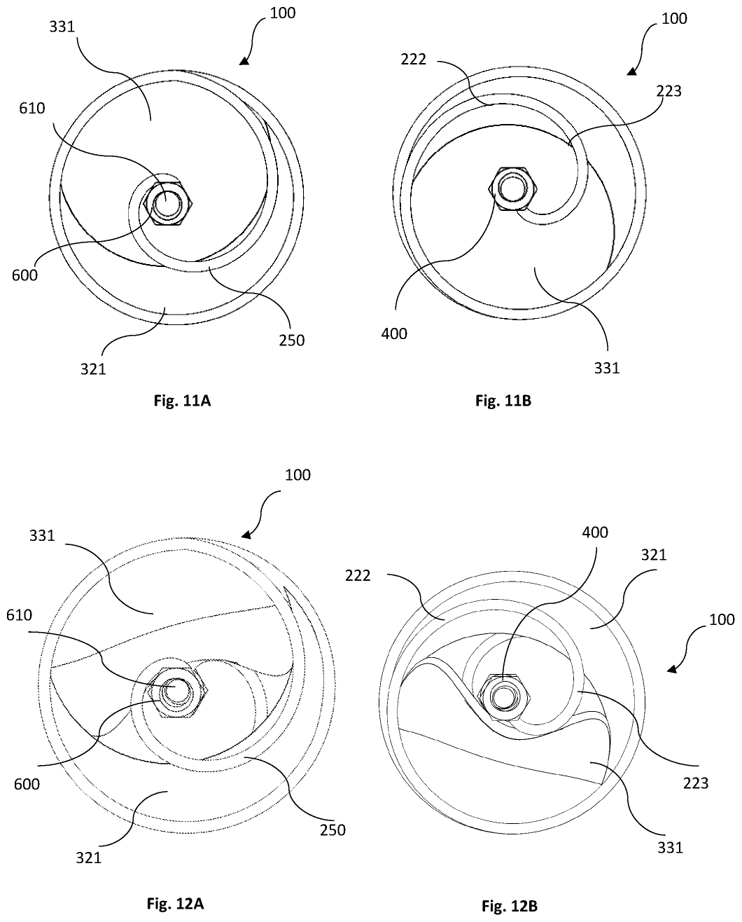

The leaflets 320, 330 are configured to move between a closed position and an open position. In the closed position, the leaflets 320, 330 extend in substantially parallel planes, such that the overlapping of the leaflets 320, 330 closes the central opening 230 to inhibit fluid flow therethrough. When implanted in a heart, during diastole, a body portion 331 of the gibbous-shaped second leaflet 330 moves in a ventricular direction into the open position to allow fluid to flow through the valve from the atria into the ventricle 100. The closed and open positions of the leaflets 320, 330 are shown in FIGS. 11 and 12, respectively. The crescent-shaped first leaflet 320 may be stiffer than the gibbous-shaped second leaflet 330 and, accordingly, may not move in the ventricular direction to as great an extent as the gibbous-shaped second leaflet 330. In some embodiments, the stiffer first leaflet 320 may not move to any substantial extent and may be considered substantially fixed relative to the frame 200. In such embodiments, the first leaflet 320 may function primarily as a backstop for the second leaflet 330.

The leaflets 300, (or 320, 330, 300a-c) may be formed from organic tissue, such as bovine or porcine leaflet tissue for example. Additionally or alternatively, one or more of the leaflets may be formed from a polymeric material (such as PTFE, for example). In some embodiments, a polymeric material may be used in combination with a reinforcing material, such as a mesh. In some such embodiments, the crescent-shaped first leaflet 320 may be formed from a polymeric material in combination with a reinforcing mesh (such as a nitinol mesh). This may strengthen the crescent-shaped first leaflet 320 to enhance inhibition of prolapse of the gibbous-shaped second leaflet 330.

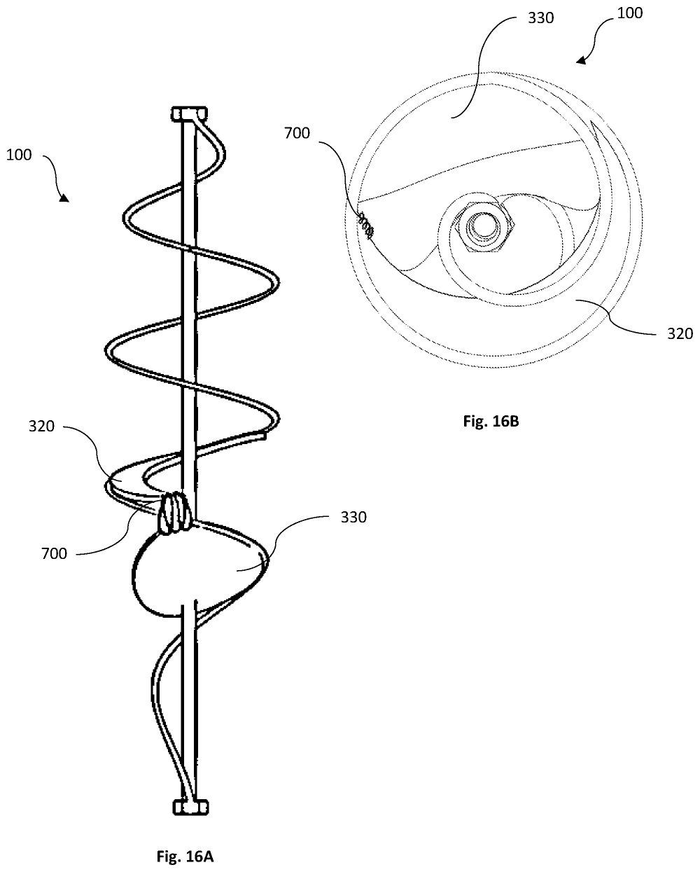

In some embodiments, the adjacent leaflets may be sutured together along a coaptation line. The coaptation line may have a length of, for instance, 2 mm. One example of a coaptation line is shown in FIGS. 16A and 16B. Sutures 700 join the first leaflet 320 and second leaflet 330 in a coaptation line adjacent the body 210 of the valve 100.

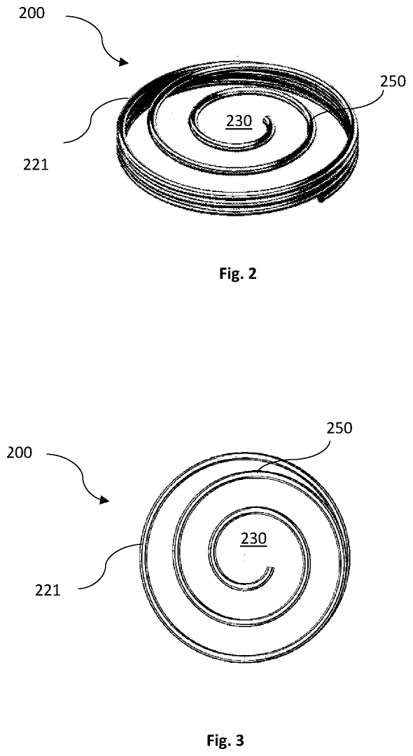

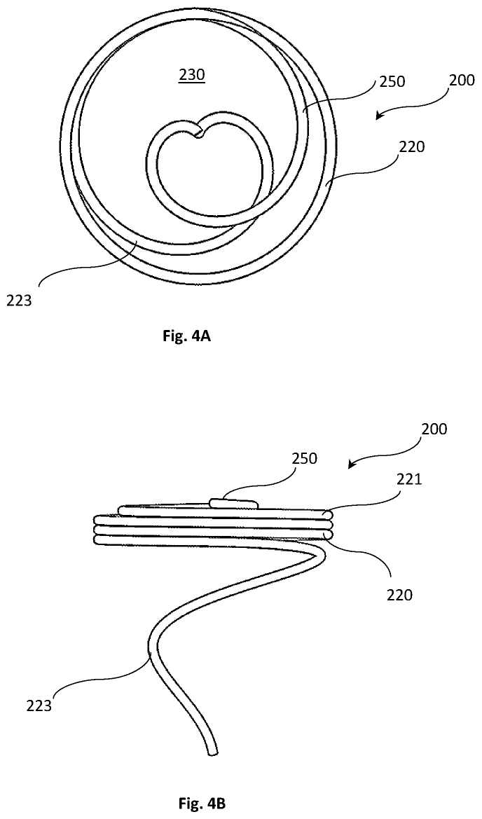

In some embodiments, the frame 200 further comprises a reinforcing member 250 extending into the central opening 230 from a perimeter of the body 210. The reinforcing member 250 is positioned abutting an atrial side of the leaflets 300. As shown in Figure. 3, in this embodiment, the reinforcing member 250 is a continuation of a superior turn of the helical coil 220 and extends substantially within a plane defined by a superior turn 221 of the helical coil 220 (that is, a proximal-most turn of the coil 220 on the atrial side of the valve 100). In other embodiments, for example as shown in FIG. 4B. the reinforcing member may extend slightly beyond superior turn 221 of the helical coil 220 in an atrial direction. For example, the reinforcing member 250 may be made from the same Nitinol wire as the helical coil 220. In this embodiment, the reinforcing member 250 has a gradually reducing diameter with each 90 degree arc. A non-linearly decrementing diameter, similar to a Fibonacci spiral, is shown in FIGS. 4A and 4B. Alternatively, a linearly decrementing diameter may be employed, such as shown in an alternative configuration of the frame 200 in FIGS. 2 and 3. In other embodiments, other ratios of decrementing arc diameter may be used. As such, the reinforcing member 250 spirals inwardly from the perimeter of the central opening 230. The reinforcing member 250 may terminate at a substantially central portion of the central opening 230, for example as shown in FIG. 4A.

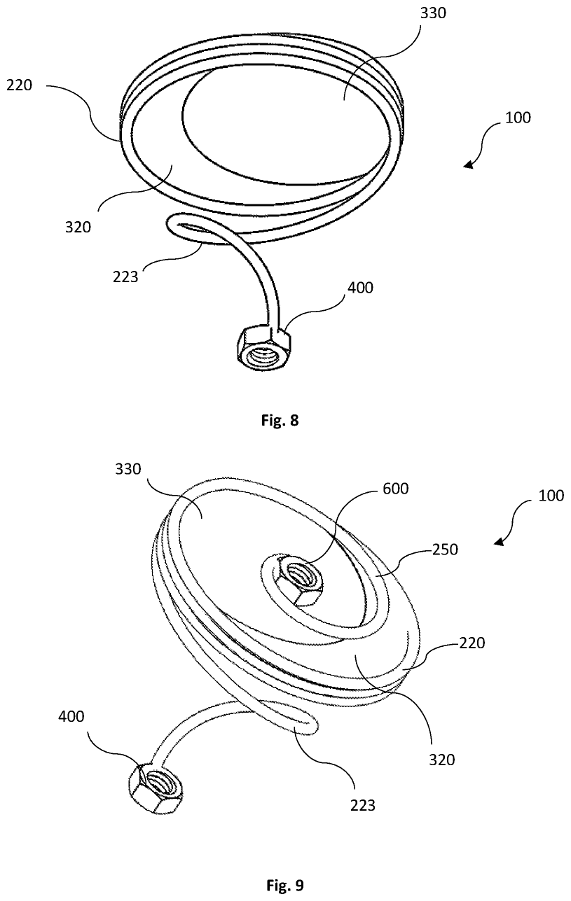

The reinforcing member 250 functions to inhibit prolapse of the leaflets 300 beyond the body 210 in an atrial direction. For example, during systole, the leaflets 300 are subjected to substantial fluid backpressure due to ventricular contraction. The reinforcing member 250 provides added support to the leaflets 300 to resist movement of the leaflets 300 in the atrial direction under this pressure, inhibiting regurgitation of blood into the atrium. As shown in FIGS. 8 and 9, in some embodiments, the valve 100 further includes an attachment member 400 for attaching the valve to a delivery device 500. Attachment to the delivery device 500 axially locks the attachment device 400 relative to the delivery device 500. In the illustrated embodiments, the helical coil 220 includes an arm 223 extending from a distal end of the helical coil 220, on the ventricular side of the valve 100. The attachment member 400 may be provided at a distal end of the arm 223. In a similar manner to the reinforcing member 250, the arm 223 of the helical coil 220 may have a reducing diameter, such that a distal portion spirals inwards and the attachment member 400 is positioned substantially centrally within the central opening 230. The arm 223 may have a linearly decrementing diameter, a decrementing diameter similar to a Fibonacci spiral, or other ratios of decrementing arc diameter may be used. The arm 223 may extend distally from the coil 220, such that attachment member 400 is positioned to allow opening of that the distal-most leaflet (such as the gibbous leaflet 330) into the ventricle. As such, the arm 223 and attachment member 400 may not obstruct opening of the leaflets 300 into the ventricle.

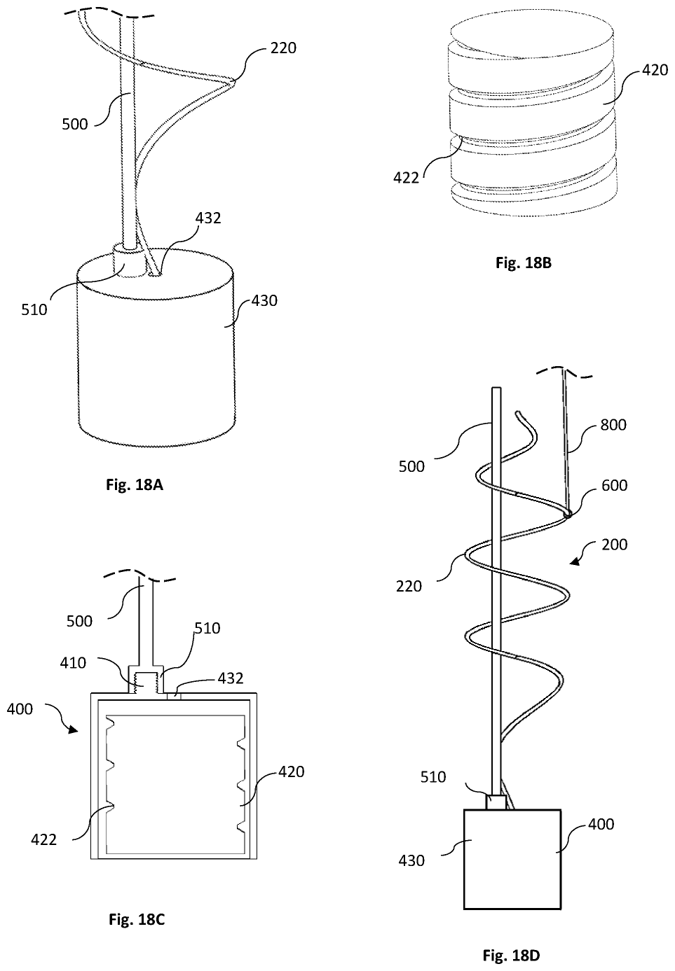

The attachment member 400 may be fixedly attached to the helical coil 220. For example, the attachment member 400 may be welded or otherwise bonded to the helical coil 220. Alternatively, a distal portion of the helical coil 220 (e.g. distal portion of the arm 223) may be interlocked with the attachment member 400. For example, the attachment member 400 may comprise a body 420 defining a recess, bore, notch or groove which is able to secure the distal portion of the helical coil 220. In the example of FIGS. 18a-d, the body 420 of the attachment member is configured to facilitate attachment of the helical coil by winding the distal portion of the helical coil 220 around a portion of the body 420. In the illustrated embodiments, the body 420 is substantially cylindrical and comprises a groove 422 extending around the body 420. The groove 422 may be helical and may extend one or more turns around the body 420 of the attachment member 400. The groove 422 may receive the distal portion of the helical coil 220, such that the distal portion of the helical coil 220 may be wrapped around and secured to the body 420.

The attachment member 400 may further comprise a case 430. The case may be configured to at least partially enclose the body 420 to cover the groove 422 and the distal end of the helical coil 220 received in the groove 422. The case 430 may slide over the body 420 as a sheath. The case 430 may include an opening 432 through which the helical wire 220 may extend, such the distal portion of the helical coil 220 is positioned within the case 430, while the remaining length of the helical coil 220 is located outside of the case 430. The case 430 may be formed from a metal, such as cobalt chrome, for example.

The attachment member 400 may include an threaded portion 410, configured to engage a correspondingly threaded portion 510 of the delivery device 500. In some embodiments, for example as shown in FIGS. 15A and 15B, the threaded portion 410 may comprise an internal thread, while the delivery device 500 comprises a corresponding external thread. In other embodiments, however, the reverse arrangement may be employed. That is, the attachment member 400 may have an external thread while the delivery device 500 has an internal thread. For example, as shown FIGS. 18a-18d, threaded portion 410 of the attachment member 400 may comprise an external thread, while the threaded portion 510 of the delivery device 500 may comprise an internal thread. The threaded portion 410 of the attachment member 400 may be provided on a proximal region of the attachment member 400, such as on a proximal region of the case 430, for example as shown in FIGS. 18a, 18c and 18d. The delivery device 500 may be releasably attachable to the attachment member 400 by rotation of the delivery device to engage the threads of the respective threaded portions 410, 510. As such, the delivery device 500 may engage the attachment member during delivery of the valve 100 and disengage the attachment member 400 after completion of delivery of the valve 100 to enable retraction of the delivery device 500. In other embodiments, the delivery device may engage the attachment member by other suitable means.

In some embodiments, the valve 100 further includes an engagement portion 600. The engagement portion 600 may be provided on the helical coil 220, for example at or adjacent to a proximal end 222 of the helical coil 220. For example, the engagement portion 600 may be provided on a terminal end of the reinforcing member 250, as shown in FIG. 1, for example. In other embodiments, the engagement portion 600 may be positioned elsewhere along the helical coil, such as intermediate a proximal and distal end of the helical coil, for example as shown in FIG. 18D. The engagement portion 600 may be configured to be engaged by one or more threads, sutures or wires (for example, silk thread 800 as shown in FIG. 18D). As shown, the engagement portion is a nut similar to the attachment member. However, in other embodiments, the engagement portion may be a loop, hook, aperture, latch or catch configured to engage the one or more sutures, threads or wires by looping the threads, sutures or wires through or around the engagement portion. When the delivery device 500 is attached and axially locked to the attachment member 400, tensile retraction force may be applied to the engagement portion 600 via the one or more sutures, threads or wires to retract the proximal end 222 of the coil, thereby to configure the valve in the axially elongated configuration for delivery to the heart, for example as shown in FIGS. 15A and 15B.

In some embodiments, the engagement portion 600 includes an aperture 610, configured to receive the delivery device 500 to align the valve 100 relative to the delivery device 500. The aperture 610 further functions as an attachment point for one or more retraction threads, sutures or wires, which are used to apply force to the engagement portion 600 to retract the proximal end 222 of the coil 220. The retraction threads, sutures or wires may have a length sufficient to allow for retractile force to be applied from outside the body as the device is delivered to the heart. In some embodiments, the length of the retraction threads, sutures or wires may be about 2 metres.

In some embodiments, the retraction may be performed in a cold environment, such as in an ice water bath, below the transformation temperature of the shape memory material, to allow the coil 220 to remain in the elongated configuration without continual application of retractile force. The loose ends of the suture, thread or wire may be kept taught and clamped onto the delivery rod so as to maintain passive retraction force when the valve is subjected to body temperature. At body temperature, the coil 220 will again be biased into the collapsed configuration.

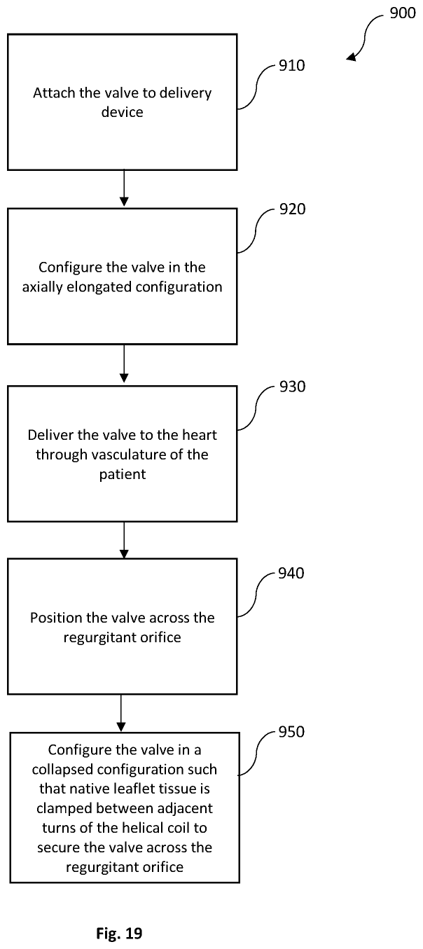

A method of delivering the device according to some embodiments of the present disclosure is described with reference to flow diagram 900 FIG. 19. While this example refers primarily to treatment of a regurgitant tricuspid valve, it will be appreciated that the described delivery techniques may be applicable for delivery of the device to a regurgitant mitral valve.

A valve 100 according to the present disclosure may be delivered to a patient's heart for repair of a regurgitant orifice between an atrium and a ventricle. The valve 100 is configured to be delivered to a native valve of the heart in the elongated configuration. Once positioned across the regurgitant orifice, the resilient bias of the coil 220 allows the coil 220 to return to its collapsed configuration once the retractile force from the suture/Nitinol thread is released. The helical coil 220 clamps onto native leaflet tissue of the heart when released to the collapsed configuration. The valve 100 thus contracts in an axial direction to secure the valve 100 to the native heart tissue. This is in contrast to many existing TMTT therapies, which utilise a radially expansile valve, in combination with a peripheral anchor system, to replace the entire annulus of a diseased valve.

Clamping of the valve tissue between the turns of the coil 220 positions the valve across the regurgitant orifice of the native valve. At present, no other TMTT device specifically targets regurgitant orifice replacement.

In some embodiments of the present disclosure, the valve 100 may be provided in a kit. The kit may include the valve 100 and the delivery device 500. The valve 100 may be sterilised. The valve 100 may be provided in a preserving medium, such as formaldehyde. Formaldehyde may maintain the sterility of the valve and inhibit degradation of the leaflets 300 (or 320, 330, 330a-c). The valve 100 may be stored at under 23° C. The kit may further include the one or more sutures or wires. In some embodiments, the kit may further include one or more of the steerable sheath, delivery rod, surgical implements, surgical consumables or other items required for completing delivery of the valve 100 to the patient's heart.

A method for implanting a valve 100 according to the present disclosure is shown in the flow diagram 900 of FIG. 19. The valve 100 may be delivered to the heart using a Transcatheter delivery method. Under general anaesthesia (and, optionally, trans-oesophageal echo guidance), a sheath is inserted into the right femoral vein. An initial delivery sheath for venous access may be a 18 Fr sheath, for example, which may be about 1 m in length. The sheath may be steerable. A rotational tool may be provided to allow the operator to flex the distal end of the sheath within the body. A guidewire may first be passed into the right ventricle and the sheath advanced over the guidewire into the right ventricle of the patient's heart. In alternative embodiments, a jugular sheath may be percutaneously inserted in the right internal jugular vein.

In step 910 of flow diagram 900, the valve 100 is attached to the delivery device 500. The valve may be attached to the delivery device 500 by inserting the delivery device 500 through the aperture 610 of the engagement device and through the central opening 230 of the valve 100, then screwing the threaded portion 510 of the delivery device 500 into the threaded portion 410 of the attachment member 400. One or more sutures or wires may be attached to the engagement portion 600, for example by threading or looping through the engagement portion 600. The loose ends of the thread may be fixed on the delivery rod outside of the patient's body for the operator to release when the device is appropriately positioned across the valve.

In step 920 of flow diagram 900, the valve 100 may then be configured in the axially elongated configuration, for example by applying retractile force to the engagement portion 600 via the one or more sutures or wires to retract the proximal end 222 of the coil 220. As described above, this step may be performed in a low-temperature environment such as an ice bath.

In step 930 of flow diagram 900, the valve 100 may be delivered to the heart through vasculature of the patient. Once in the axially elongate position, the valve 100 may be advanced through the sheath and through the vasculature of the patient. For example, the valve 100 may be advanced through the femoral vein, up the inferior vena cava and across the tricuspid valve regurgitant orifice. The steerable sheath may be used to direct the delivery device 500 and the valve 100 across the tricuspid valve regurgitant orifice.

Delivery to the mitral valve may be achieved using a similar process, with the additional step of a trans-septal puncture to gain access into the left atrium. The steerable sheath would be placed in the left ventricle and directed towards the mitral valve regurgitant orifice.

Intravenous heparin would be required during this procedure to keep the activated clotting time (ACT) between 250-300 seconds.

As indicated in step 940 of flow diagram 900, the valve 100 may be positioned across the regurgitant orifice. The valve 100 is then positioned across the regurgitant orifice with the valve leaflets on the ventricular side of the regurgitant orifice.

The operator may remove the thread/wire clamp and gently reduce the retraction force of the proximal end 222 of the coil 220, allowing the resilient bias of the coil 220 to return the valve 100 to the collapsed configuration. As the valve 100 returns to the collapsed configuration, native leaflet tissue is clamped between adjacent turns of the helical coil 220, securing the valve 100 across the regurgitant orifice, as indicated in step 950 of flow diagram 900. In some embodiments, the valve may be configured to clamp the native valve tissue between the first and second turns of the coil on the atrial side of the valve. As the frame 200 returns to the collapsed configuration, the leaflets (for example, leaflets 300, leaflets 320 and 330 or leaflets 300a, 300b and 300c) are sequentially deployed into position to substantially cover the regurgitant orifice.

Valves according to embodiments of the present disclosure may provide a way to perform lower risk mitral or tricuspid valve repair, to improve patient symptoms and prolong life. Current Transcatheter Mitral and Tricuspid Therapies (TMTT) employ devices which are difficult to use and involve long procedural times, while only providing a small improvement in patient outcomes. Valves according to embodiments of the present disclosure may provide greater efficiency than conventional TMTT devices. This valve may also be simpler to use, provide targeted regurgitant orifice replacement, reduce procedure time and/or improve patient outcomes.

Further, valves according to the present disclosure may also be applicable to a greater range of indications than conventional therapies. For example, valves according to the present disclosure may be applicable for repair (or replacement) of native regurgitant aortic valves or pulmonary valves.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A valve for repair of a regurgitant orifice between an atrium and a ventricle in a heart, the valve comprising:
a frame comprising a helical coil having at least two turns and defining a central opening; and
one or more leaflets attached to the helical coil and extending into the central opening,
wherein, when the valve is implanted, the valve is configured to clamp native leaflet tissue of the heart between adjacent turns of the helical coil.

2. The valve of claim 1, wherein the helical coil is formed from a shape memory material.

3. (canceled)

4. The valve of claim 1, wherein the frame is configurable between an axially elongated configuration and a collapsed configuration.

5. The valve of claim 4, wherein, in the collapsed configuration, the helical coil comprises an annular body with a first diameter and wherein in the axially elongated configuration, the diameter of the annular body defined by the helical coil is reduced by about 50% or more relative to the collapsed configuration.

6. The valve of claim 4, wherein the coil is resiliently biased into the collapsed configuration.

7. The valve of claim 6, wherein the valve is configured to be delivered to a native valve of the heart in the elongated configuration and configured to clamp native leaflet tissue of the heart when released to the collapsed configuration.

8. (canceled)

9. The valve of claim 1 comprising a first leaflet and a second leaflet.

10. The valve of claim 9, wherein the first leaflet and the second leaflet are unequal in size.

11. The valve of claim 9, wherein the first leaflet has a crescent shaped portion extending into the central opening.

12. The valve of claim 11, wherein the second leaflet has a gibbous shaped portion extending into the central opening.

13. (canceled)

14. (canceled)

15. The valve of claim 9, wherein the first and second leaflets overlap within the central opening.

16. (canceled)

17. (canceled)

18. The valve of claim 15, wherein the first leaflet inhibits movement of the second leaflet beyond the body in the atrial direction.

19. The value of claim 1 comprising three leaflets.

20. (canceled)

21. The valve of claim 1, further comprising a reinforcing member extending into the central opening from a perimeter of the body on an atrial side of the leaflets, for inhibiting prolapse of the leaflets beyond the body in an atrial direction.

22. The valve of claim 21, wherein the reinforcing member is a continuation of a superior turn of the helical coil.

23. The valve of claim 22, wherein the reinforcing member has a reducing diameter such that the reinforcing member spirals inwardly from the perimeter of the central opening.

24. (canceled)

25. The value of claim 1, further comprising:
an attachment member for attaching the valve to a delivery device; and
an engagement portion,
wherein, when the delivery device is attached to the attachment member, tensile force may be applied to the engagement portion to retract the proximal end of the coil, thereby to configure the valve in an axially elongated configuration for delivery to the heart.

26-31. (canceled)

32. A kit, comprising:
a valve according to claim 1; and
a delivery device, wherein the delivery device comprises and elongate rod having an attachment member at a distal end, the attachment member configured to attach to a distal end of the helical coil.

33. (canceled)

34. A method of delivering a valve according to claim 1 for repair of a regurgitant orifice between an atrium and a ventricle in a patient's heart, the method comprising:
attaching the valve to a delivery device;
configuring the valve body in an axially elongated configuration;
delivering the valve to the heart through vasculature of the patient;
positioning the valve across the regurgitant orifice with the valve leaflets on the ventricular side of the regurgitant orifice; and
configuring the valve in a collapsed configuration such that native leaflet tissue is clamped between adjacent turns of the helical coil to secure the valve across the regurgitant orifice.

35. (canceled)

36. The method of claim 34, wherein the delivery device and the valve are delivered to the patient's heart via a steerable catheter.

37. (canceled)

38. (canceled)
